(19) United States
(12) Plant Patent Application Publication
(10) Pub. No.: US 2002/0112262 P1
(43) Pub. Date: Aug. 15, 2002

(54) Miniature rose plant named 'BENmfig'

(75) Inventor: Frank Benardella, Englishtown, NJ (US)

Correspondence Address: John M. Saville, Nor' East Miniature Roses, Inc., P.O. Box 307, Rowley, MA, 01969-0607, US

(73) Assignee: Nor'East Miniature Roses, Inc., Rowley, MA (US)

(21) Appl. No.: 09/783,455

(22) Filed: Feb. 14, 2001

(51) Int. Cl. A01H 5/00

(52) U.S. Cl. PLT/121

(57) ABSTRACT

BENmfig is a new and distinct variety of miniature rose plant primarily identified by it's fragrant, pink blend buds and flowers of hybrid tea form. The extremely vigorous plant bears a continuous display of these long lasting flowers on stems up to 9 inches in length, most often borne singly but also in loose clusters, making it excellent as a cut flower. The dark olive green foliage stands in contrast to the blooms. This plant can be grown both in a greenhouse and outdoors as a garden perennial or decorative specimen plant, requiring winter protection in cooler climates.

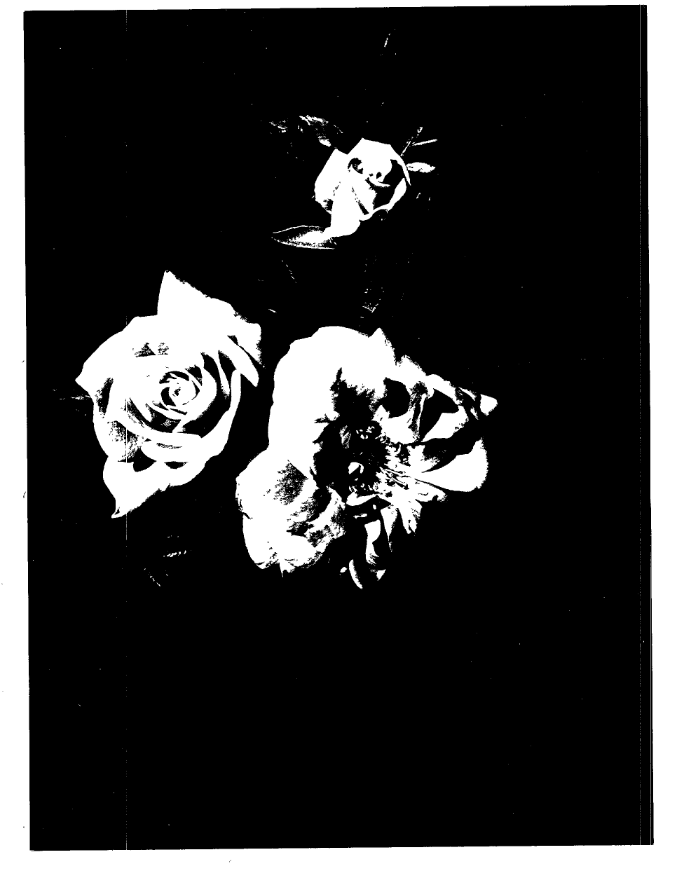

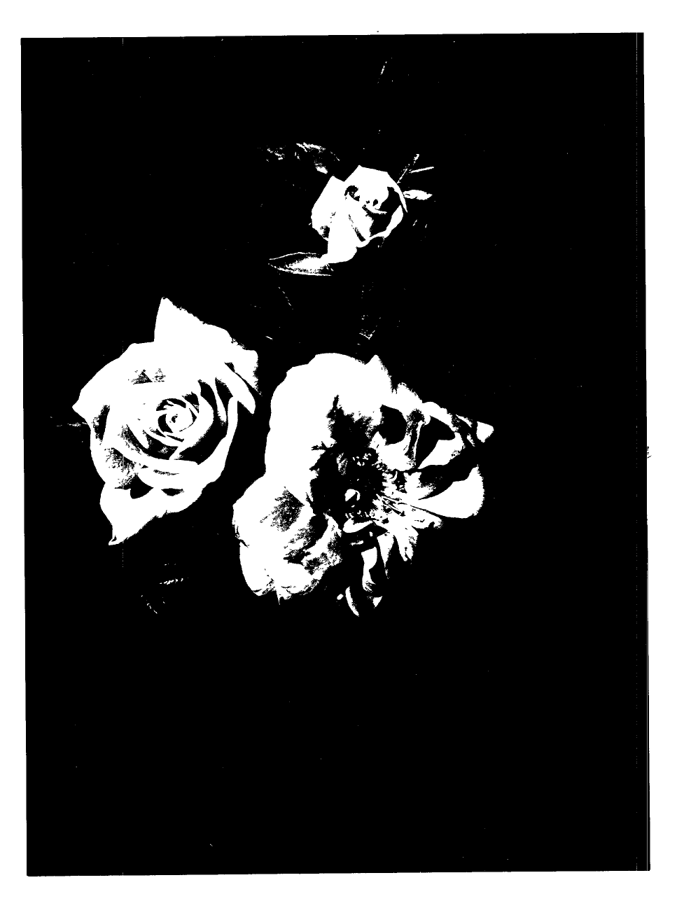

### CROSS REFERENCE

This present invention relates to a new and distinct variety of hardy, bush type plant of the miniature rose class (rosa chinensis minima), with some resemblance to its seed parent, BENfig (U.S. Plant Pat. No. 8,020), having a similar bud shape and petal count. The differences between the two plants are that the flowers of this new invention are darker and more variable shades of pink and the plant of this new invention is more vigorous and somewhat larger. Both plants bear flowers on long stems for cutting.

### BACKGROUND OF THE INVENTION

This present variety of miniature rose was created in a greenhouse in Englishtown, N.J. by crossing BENfig (U.S. Plant Pat. No. 8,020), a light pink, fragrant miniature rose, as the seed parent and BENmagic (U.S. Plant Pat. No. 8,603) a red and white miniature rose.

Asexual reproduction by cuttings of this new variety shows that all distinguishing characteristics of this rose continually come true to form.

### SUMMARY OF THE INVENTION

The present invention relates to a new and distinct variety of hardy dwarf bush type rose plant of the miniature class; the variety being primarily characterized—as to novelty—by it's buds and flowers of white and light, medium and dark pink coloring, and which are generally of exhibition (hybrid tea) form.

The variety is further characterized by:

A continuous display of buds and flowers, most often borne singly on long stems but also in loose clusters making it excellent for cutting.

A strong rose fragrance.

A well branched and upright plant having a mature height of 30 inches and becoming 30 inches or more in width.

A plant with extreme vigor, regrowing very quickly to it's 30 inch height after winter dieback or after being cut back at any time during the growing season.

Dark green, semi-glossy foliage.

A plant which grows and blooms in a greenhouse or outdoors as a garden perennial, decorative specimen plant or just for cut flowers.

### BRIEF DESCRIPTION OF THE DRAWING

The accompanying color photograph show specimens of the flower of this new variety in different stages of development as grown and observed in October, under plastic in Essex County, Mass.

### BOTANICAL DESCRIPTION OF THE PLANT

Following is a detailed description in outline form pertaining specifically to this new and distinct variety of miniature rose plant. All major color plate identifications made are referring to The Royal Horticulture Society Chart except where common terms of color definition are employed.

Class: Miniature rose

### FLOWERS

Blooming habit: continuous

Borne: mostly singly on stems up to 9″ in length, but also in loose clusters.

Bud:

Size.—medium, averaging ⅞ inch in length.

Form.—ovate.

Color.—When sepals first divide—a dark rose pink, near 55A.

Sepals.—color—medium yellow green, between 146A and 144A. characteristics—lanceolate with smooth edges and occasionally with small “flags”; extending well beyond the tip of the bud, rolling back to the stem as the petals unfurl, and dropping with the petals.

Peduncle.—length—averaging 1 inch aspect—upright strength—erect color—a medium yellow green, near 146B.

Bloom:

Size.—When fully expanded—up to 2 inches.

Form.—High centered at first, edges of petals remaining loosely curled inward as the bloom starts to open, then outer petals start to loosely roll outward from the tip and remaining thus until dropping.

Petalage.—averaging 25.

Petaloids.—usually 1 to 5.

Fragrance.—strong rose.

Petals:

Texture.—medium thick.

Appearance.—Inside and outside—satiny.

Form.—broad spatulate.

Arrangement.—Arranged shingle-like.

Color.—as petals begin to unfurl, a light pink, near 62D flushed lightly with a darker pink, near 63B, and the reverse is a very soft pink, near 49D. During the first two days—a medium pink, near 55C and the centers of the petals still flushed lightly with near 63B. When half blown—the color lightens a little to near 55D and all but the very edges are flushed with near 55C. The base of each petal is a very light yellow, near 11D, and the reverse is still soft pink, near 49D. As the bloom ages it lightens in color to near 49D but the flushing intensifies to near 54D blending out from the center of each petal and with near white blending up from the base of the petals. The very innermost petals and petaloids are often flushed an even deeper pink, near 63A. The reverse of the petals is still 49D but at this stage becomes flushed with near 55B. (The intensity of the flushing only, on the entire bloom and at all stages, increases proportionately with the amount of direct sunlight and coolness of temperature).

General Tonality.—a combination of light, medium and dark pinks.

General Tonality from a distance.—medium pink.

### REPRODUCTIVE ORGANS

Stamens, filaments and anthers:

Arrangement.—regularly arranged around styles.

Quantity.—ample.

Filaments.—length—varying from ⅛ inch to ¼ inch. general color—white

Anthers.—color—a very light yellow, near 13D.

Pollen:

Color.—medium yellow, near 16B.

Quantity.—some.

Pistils, styles and stigmas:

Quantity.—some.

Styles.—thin, uneven length, to about ⅛ inch. color—a very pale primrose yellow, near 4D.

Stigmas.—a pale orange yellow, near 20C.

Hips:

Shape.—globular.

Size.—averaging just over ½ inch in diameter (17/32 inch).

Color.—orange, near 169C and 169D.

### PLANT

Habit: upright to 30 inches and 30 inches or more wide near the top, uniform.

Growth: extremely vigorous and well branched.

Foliage:

Size.—medium.

Quantity.—abundant.

Color.—New foliage—dark green on the upper surface, between 146A and 137C, and flushed reddish, near 182A; and the underside a dark olive green, near 146A, and also flushed reddish. Older foliage—Upper surface—a dark olive green, near 147; underside—medium olive green, near 147B.

Texture.—Upper surface—semi-glossy; underside, matte.

Leaflets.—5 or 7 leaflets and occasionally 3 shape—oval, pointed edge—medium-fine serration.

Leaf stem.—olive green, near 146A, and the underside of the leaf stem being a lighter olive green, near 146B.

Stipules.—averaging ⅞ inch in length.

Resistance: average resistance black spot and downy mildew, no rust observed.

Wood: Smooth with new wood being a medium yellow green, near 144B, and old wood a medium olive green, near 147B.

Thorns:

Quantity on main stalks and laterals.—ordinary.

Form.—a flat base tapering to a fine point, thin, straight and being slightly angled downward, and being of medium length.

Color when young.—a pale yellow green flushed with light coral.

Winter Hardiness: Needs protection in cooler climates.

1. I claim a new and distinct variety of rose plant of the miniature rose class, substantially as illustrated and described, characterized particularly by its fragrant, exhibition form buds and flowers of varying pink tonality, being borne on long stems on an extremely vigorous plant making it excellent for use for cut flowers.
